(19) United States
(12) Patent Application Publication
(10) Pub. No.: US 2007/0203746 A1
(43) Pub. Date: Aug. 30, 2007

(54) System and user interface enabling user order item selection for medical and other fields

(75) Inventors: Jan DeHaan, Downingtown, PA (US); Gary G. Hardel, Wayne, PA (US); Randall Case, Blue Bell, PA (US); Zhijing Liu, Audubon, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS HEALTH SERVICES CORPORATION, MALVERN, PA (US)

(21) Appl. No.: 11/549,346

(22) Filed: Oct. 13, 2006

Related U.S. Application Data
(60) Provisional application No. 60/729,527, filed on Oct. 24, 2005.

(51) Int. Cl. G06Q 10/00; A61B 5/00

(52) U.S. Cl. 705/2; 600/300

(57) ABSTRACT

A user interface system reduces the number of user interactions required in order data entry by providing data representing a candidate order for provision of an item or services including user modifiable values of a set of related attributes and provides further candidate alternatives in response to user selection of an attribute value and other heuristics. A user interface system enabling user selection of related order parameters identifying an order for providing an item incorporates a repository including information identifying candidate items for order and associated corresponding related order parameters. An individual item for order is associated with multiple related order parameters. A user interface processor provides data representing a display image identifying an initial complete candidate order including multiple related order parameters, in response to user entry of order associated data. In response to user selection of a first order parameter, having a first value, of the initial complete candidate order, the user interface processor provides data representing multiple different individually user selectable complete candidate orders individually incorporating values of the first order parameter excluding the first value, the complete candidate orders being derived using the repository and based on user ordering history. The user interface processor selects one of the multiple different individually user selectable complete candidate orders in response to user command.

### CROSS-REFERENCE TO RELATED APPLICATIONS

The present application is a non-provisional application of provisional application having Ser. No. 60/729,527 by J. DeHaan et al. on Oct. 24, 2005.

### FIELD OF THE INVENTION

The present invention concerns a user interface system enabling user selection of related parameters in constructing an order for providing an item such as a service or medication for administration to a patient.

### BACKGROUND OF THE INVENTION

Existing Physician order entry or documentation systems for ordering a service or medication for provision to a patient, for example, require a user to make multiple selections and/or re-evaluate changes made by the system in response to user actions in ordering an item. Existing systems typically present sets of order related attribute values, concerning the nature of an order, as separate data fields or as a single candidate phrase or phrase representing an order for an item. When a user changes one attribute in the set, some prior systems do not change the value of the other related attributes. Other prior systems do change one or more values in the set, but do not display more than one set. Some of these prior systems merely highlight changes made by the system.

Some existing systems do not change attribute values in response to a user action and do not require a user to select each attribute value needed to be changed and to select a new value for that attribute from a list. Other existing systems respond to a change of a single attribute by displaying a new single candidate set of attribute values or replace a current set with a new set. Further existing systems highlight system generated changes to counteract the user tendency to not re-evaluate attribute values they have previously viewed and deemed correct. Other existing systems present combinations of attribute values in a single menu list to allow the user to set multiple values with a single selection, but these combinations often represent only part of an attribute set (or candidate phrase) and not the entire set or phrase.

Existing systems that do react to user actions by changing one or more attribute values require the user to re-evaluate the values of attributes and if the new candidate set is not correct, the user needs to make at least one other change. In addition, some users tend to not re-evaluate attribute values they have looked at before and deemed correct, even if the system changed one or more of these viewed values in response to a user action. Highlighting of such system generated changes may reduce this user tendency, hut does not eliminate it. A system according to invention principles addresses these deficiencies and associated problems.

### SUMMARY OF THE INVENTION

A user interface system provides data representing a candidate order for provision of an item or service including user modifiable values of a set of related attributes and provides further candidate order alternatives in response to user selection of an attribute value and other heuristics. A user interface system enabling user selection of related order parameters identifying an order for providing an item incorporates a repository including information identifying candidate items for order and associated corresponding related order parameters. An individual item for order is associated with multiple related order parameters. A user interface processor provides data representing a display image identifying an initial complete candidate order including multiple related order parameters, in response to user entry of order associated data. In response to user selection of a first order parameter, having a first value, of the initial complete candidate order, the user interface processor provides data representing multiple different individually user selectable complete candidate orders individually incorporating values of the first order parameter excluding the first value, the complete candidate orders being derived using the repository and based on user ordering history. The user interface processor selects one of the multiple different individually user selectable complete candidate orders in response to user command.

### BRIEF DESCRIPTION OF THE DRAWINGS

FIG. 1 shows a system supporting ordering of an item, in accordance with invention principles.

FIG. 2 shows a flowchart of a process performed by a system supporting ordering of an item, in accordance with invention principles.

FIGS. 3-9 illustrates user interface image menus provided by the system supporting ordering of an item, in accordance with invention principles.

### DETAILED DESCRIPTION OF THE INVENTION

A user interface system provides data representing a candidate order for provision of an item and facilitates user determination of values of a set of related attributes via menu selection. The attribute values may represent selection criteria for computerized data retrieval, represent values to be stored in a data repository, or may represent order parameters for other computerized functions such as clinician order entry. The system presents sets of related attribute values as individual data entry fields or as a candidate phrase. When a user changes one attribute value, the system uses the attribute value and other heuristics to generate one or more alternate candidate sets of related values or one or more alternative candidate phrases. The system reduces the number of user interactions with a computer user interface required to assign values to a set of related attributes and increases the likelihood that the user makes a single selection to obtain a desired order, thereby avoiding a need to make multiple selections and to evaluate intermediate results to arrive at this desired order.

The system reduces average total time required to create an order set for provision of an item or service or to complete a document by reducing the number of user interactions and by reducing the need for the user to read and evaluate intermediate results generated by the system. The system achieves this by using current context information (including user identifier, patient identifier, patient problem identifier, diagnosis identifier) and by recognizing that user selection of an attribute value is an indication that the user wants to change that value. The system reduces the number of user actions to navigate and select values from menus, reduces the need to reevaluate an order or document attribute value sets or candidate phrases and reduces the potential for user error caused by failure to notice changes made by the system in response to user actions.

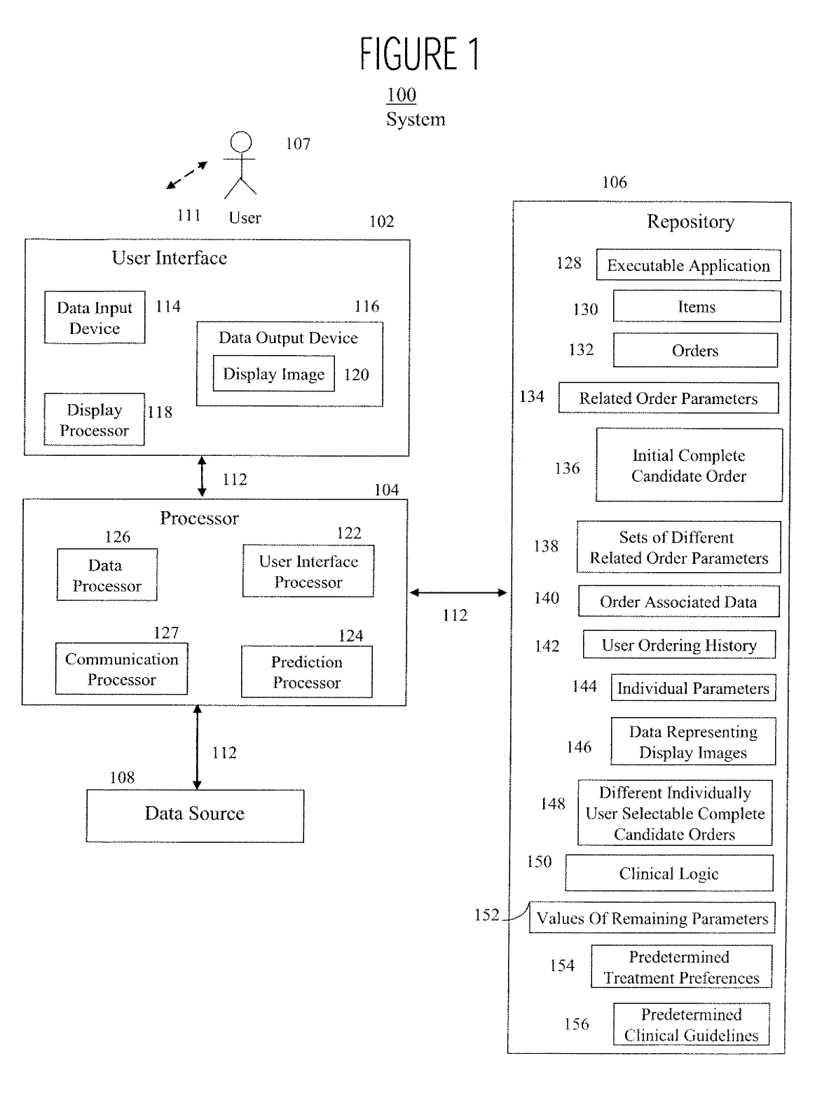

FIG. 1 shows system 100 supporting ordering of an item. The system 100 includes a user interface 102, a processor 104, and a repository 106. A user 107 and a data source 108 interact with the system 100. A communication path 112 interconnects elements of the system 100, and/or interconnects the system 100 with the data source 108. The dotted line near reference number 111 represents interaction between the user 107 and the user interface 102. The user interface 102 further provides a data input device 114, a data output device 116, ad a display processor 118. The data output device 116 further provides one or more display images 120, which are presented for viewing by the user 107. The processor 104 further includes a user interface processor 122, a prediction processor 124, a data processor 126, and a communication processor 127. The repository 106 further includes an executable application 128, items 130, orders 132, related order parameters 134, an initial complete candidate order 136, sets of different related order parameters 138, order associated data 140, user ordering history 142, individual parameters 144, data representing display images 146, different individually user selectable complete candidate orders 148, clinical logic 150, values of remaining parameters 152, predetermined treatment preferences 154, and predetermined clinical guidelines 156.

The data source 108 represents a source of any information that may be needed or used by the system 100 including, for example, any of the information stored in the repository 106. The information may be pushed to the system 100 and/or pulled by the system 100, automatically and/or manually, at one time, periodically, or as needed.

The system 100 may be employed by any type of enterprise, organization, or department, such as, for example, providers of healthcare products and/or services responsible for servicing the health and/or welfare of people in its care. For example, the system 100 represents a healthcare information system. A healthcare provider provides services directed to the mental, emotional, or physical well being of a patient. Examples of healthcare providers include a hospital, a nursing home, an assisted living care arrangement, a home health care arrangement, a hospice arrangement, a critical care arrangement, a health care clinic, a physical therapy clinic, a chiropractic clinic, a medical supplier, a pharmacy, a doctor's office, a dental office, and individual practitioners such as doctors and nurses. When servicing a person in its care, a healthcare provider diagnoses a condition or disease, and recommends a course of treatment to cure the condition, if such treatment exists, or provides preventative healthcare services. Examples of the people being serviced by a healthcare provider include a patient, a resident, a client, and an individual.

The system 100 may be fixed and/or mobile (i.e., portable). The system 100 may be implemented in a variety of forms including, but not limited to, one or more of the following: a personal computer (PC), a desktop computer, a server, a laptop computer, a workstation, a minicomputer, a mainframe, a supercomputer, a network-based device, a personal digital assistant (PDA), a smart card, a cellular telephone, a pager, a wristwatch, and a paper computer.

The system 100 and/or elements contained therein also may be implemented in a centralized or decentralized configuration. The system 100 may be implemented as a client-server, web-based, or stand-alone configuration. In the case of the client-server or web-based configurations, the executable application 128 may be accessed remotely over a communication network.

The communication path 112 (otherwise called network, bus, link, connection, channel, etc.) may use any type of protocol or data format. The protocol or data format includes, but is not limited to, one or more of the following: an Internet Protocol (IP), a Transmission Control Protocol Internet protocol (TCPIP), a Hyper Text Transmission Protocol (HTTP), an RS232 protocol, an Ethernet protocol, a Medical Interface Bus (MIB) compatible protocol, a Local Area Network (LAN) protocol, a Wide Area Network (WAN) protocol, a Campus Area Network (CAN) protocol, a Metropolitan Area Network (MAN) protocol, a Home Area Network (HAN) protocol, an Institute Of Electrical And Electronic Engineers (IEEE) bus compatible protocol, a Digital and Imaging Communications (DICOM) protocol, XML, JSON, a Health Level Seven (HL7) protocol, ASCII, and Unicode.

The user interface 102 permits bi-directional exchange of data between the system 100 and the user 107 of the system 100 or another electronic device, such as a computer or an application, for example.

The data input device 114 typically provides data to a processor in response to receiving input data either manually from a user or automatically from another electronic device. For manual input, the data input device is a keyboard and a mouse, but also may be a touch screen, or a microphone and a voice recognition application, for example.

The data output device 116 typically provides data from a processor for use by a user or another electronic device. For output to a user, the data output device 116 is a display, such as, a computer monitor or screen that generates one or more display images 120 in response to receiving the display signals from the display processor 118, but also may be a speaker or a printer, for example.

The display processor 118 (e.g., a display generator) includes electronic circuitry or software or a combination of both for generating the display images 120 or portions thereof in response to receiving data representing display images 146, which are stored in the repository 106. The data output device 116, implemented as a display, is coupled to the display processor 118 and displays the generated display images 120. The display images 120 provide, for example, a graphical user interface, permitting user interaction with the processor 104 or other device. The display processor 118 may be implemented in the user interface 102 and/or the processor 104. A display processor or generator is a known element comprising electronic circuitry or software or a combination of both for generating display images or portions thereof. A user interface comprises one or more display images enabling user interaction with a processor or other device.

The system 100, elements, and/or processes contained therein may be implemented in hardware, software, or a combination of both, and may include one or more processors, such as processor 104. A processor is a device and/or set of machine-readable instructions for performing tasks. The processor includes any combination of hardware, firmware, and/or software. The processor acts upon stored and/or received information by computing, manipulating, analyzing, modifying, converting, or transmitting information for use by an executable application or procedure or an information device, and/or by routing the information to an output device. For example, the processor may use or include the capabilities of a controller or microprocessor.

The user interface processor 122 and the prediction processor 124 perform specific functions for the system 100, as explained in further detail herein below. The data processor 126 performs other general data processing for the system 100. The communication processor 127 manages communications within the system 100 and outside the system 100, such as, for example, with the data source 108.

The repository 106 represents any type of storage device, such as computer memory devices or other tangible storage medium, for example. The repository 106 may be implemented as a database, for example. The repository 106 represents one or more memory devices, located at one or more locations, and implemented as one or more technologies, depending on the particular implementation of the system 100.

An executable application, such as the executable application 128, comprises machine code or machine readable instruction for implementing predetermined functions including, for example, those of an operating system, a software application program, a healthcare information system, or other information processing system, for example, in response user command or input.

An executable procedure is a segment of code (i.e., machine readable instruction), sub-routine, or other distinct section of code or portion of an executable application for performing one or more particular processes, and may include performing operations on received input parameters (or in response to received input parameters) and providing resulting output parameters.

A calling procedure is a procedure for enabling execution of another procedure in response to a received command or instruction. An object comprises a grouping of data and/or executable instructions or an executable procedure.

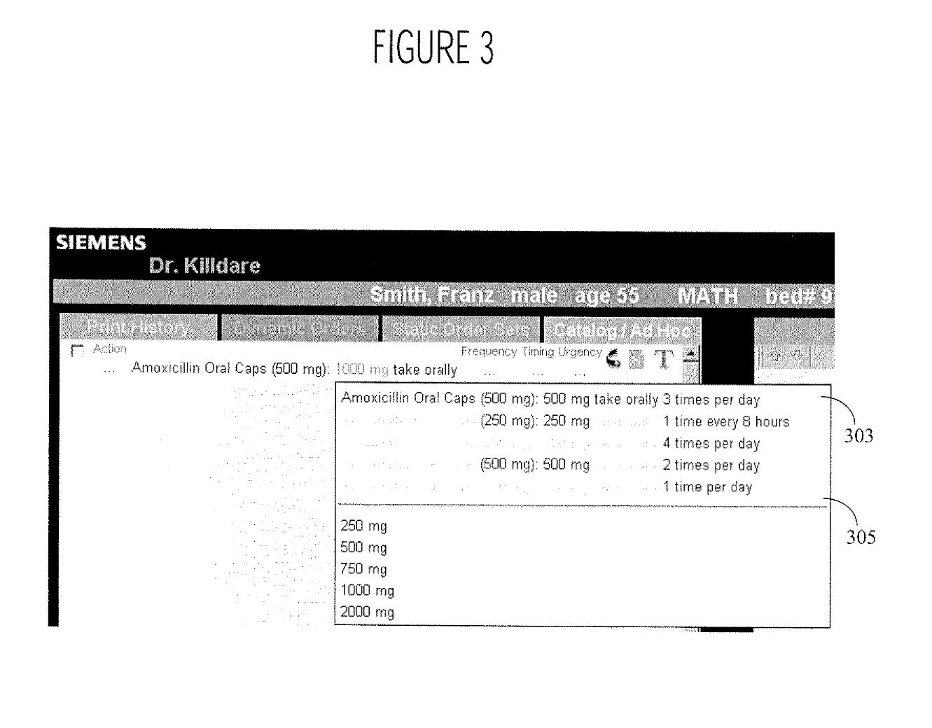

The system 100 provides a computer user interface 102 that involves the user 107 specifying the value of multiple attributes, otherwise called parameters, through menu selections, as described in FIGS. 3-9, for example. The attribute values may represent selection criteria for computerized data retrieval, represent values to be stored in the repository 106, or may represent parameters for other computerized functions.

In one embodiment, system 100 generates an initial candidate phrase (alpha-numeric text string or ideogram) and alternative candidate phrases by combining information including context information, clinical knowledge and information, patient information, user order and documentation preferences and by applying heuristic algorithms. For example, a physician desires to prescribe an antibiotic to treat a patient that has a bacterial infection. However, there are many antibiotics to choose from. System 100 checks a patient electronic medical record and finds that the patient is allergic to penicillin and that the patient is a child. The system uses clinical knowledge and information in providing an initial candidate phrase 303 as illustrated in FIG. 3 and alternative candidate phrases that describe orders for antibiotics that do not include penicillin and that have a suggested dose appropriate for children. The list of antibiotics and dosing variations is large and the system presents the top five alternatives in image window area 305 based on how often a particular antibiotic and dose have been ordered by the physician or by the institution concerned in the past. This is based on the heuristic information that if a particular antibiotic was ordered often in the past, there is a higher likelihood that the physician would want to order that same antibiotic again.

System 100 uses heuristics to determine which combination of menu items should be represented in image window area 305 (FIG. 3). Heuristics are used when the number of possible combinations of menu items is large and difficult or impractical to navigate. In computer science, heuristics relates to, or comprises use of, a problem-solving technique in which an appropriate solution of several potential solutions is found by alternative methods selected at successive stages of a program for use in the next step of the program. A heuristic is a function, h(n), defined on the nodes of a search tree, which serves as an estimate of the cost of the optimal path from that node to the goal node. Another heuristic is a function, h2 (n), defined on the end nodes of a search tree, which serves as an estimate of the utility of a path from root to end node.

System 100 includes an inference engine (prediction processor 124) employing description logic or fuzzy logic and a neural network or statistical function operating on historical data 142 together with domain (e.g. clinical) knowledge 150, 156 expressed in traditional file systems, relational or object oriented databases or native XML databases. Description logic is a way to describe in a precise way how things are organized in hierarchies of classes (e.g. penicillin is a kind of antibiotic [penicillin is a subclass of the antibiotics class]) and how classes and members of classes relate to each other (e.g. members of the class antibiotics may be used to treat members/instances of the infections class). Based on the fact that antibiotics may be used to treat infections inference engine 124 concludes that penicillin may be an appropriate choice to treat the infection. But if the patient belongs to the class of people who are allergic to penicillin, inference engine 124 concludes that penicillin is not an appropriate choice for the current patient. Fuzzy logic enables incorporation of blurry boundaries. For example, a child is any person between the age of 12 and 18. If a person is 12, she is most likely a child. If she is 18, she is most likely an adult. A 15 year old person falls somewhere in between. Assume that the system knows about drug dosing appropriate for adults and dosing appropriate for children. Using fuzzy logic the system displays just the pediatric dose when the patient is 12, just the adult dose when the patient is 18 and both doses when the patient is 15. The system may display the pediatric dose first when the patient is 14 and the adult dose first when the patient is 16.

If clinical information 150, 156 does not contain explicit information about adult and pediatric dosing, a neural network may be trained on historical patient data 140, 142 and determine that there is a relationship between patient weight and the dose of a particular drug. For example heavier patients get a higher dose. The neural net automatically determines this relationship. The programmer or user does not have to explicitly state which variables are involved. A neural network may also find that older patients get higher doses than younger patients. This information may be used by inference engine 124 to predict a dose a physician is going to order. Statistical functions are used by processor 104 to discover a mathematical relationship between patient weight and a prescribed dose of a drug. For example a linear regression analysis function applied to patient age as the independent variable and drug dose as the dependent variable may lead to the conclusion that there is a high correlation between the two variables and that the average dose is 10 milligram of drug for every 1 kilogram of patient. Again, this information is used by inference engine 124 to reliably predict what actual drug dose the physician is going to prescribe.

In conventional file systems, an executable application needs to know how to format, store and retrieve information and where data is located on a storage medium. Databases provide data in a particular format to an executable application (relational databases use tables, object oriented databases organize data based on a class an item belongs to, XML databases show tags and attributes in addition to the data and organize the data in hierarchical trees). Databases hide implementation details such as how the data is stored on a disk. They also provide additional services such as backup and recovery, indexing, transaction control (commit and roll-back), etc.

Clinical domain knowledge is expressed in terms of a vocabulary (the standard terms, words and synonyms used to indicate a clinical feature like body site [arm, leg], the relationship between terms [a hand is part of an arm], constraints on relationships [an arm can at most have one hand] and rules like “you can treat bacterial infections with antibiotics”). A variety of file and database systems may be used to store this kind of information. XML is one commonly used format and theoretically, native XML databases facilitate handling the clinical domain knowledge type of data structure.

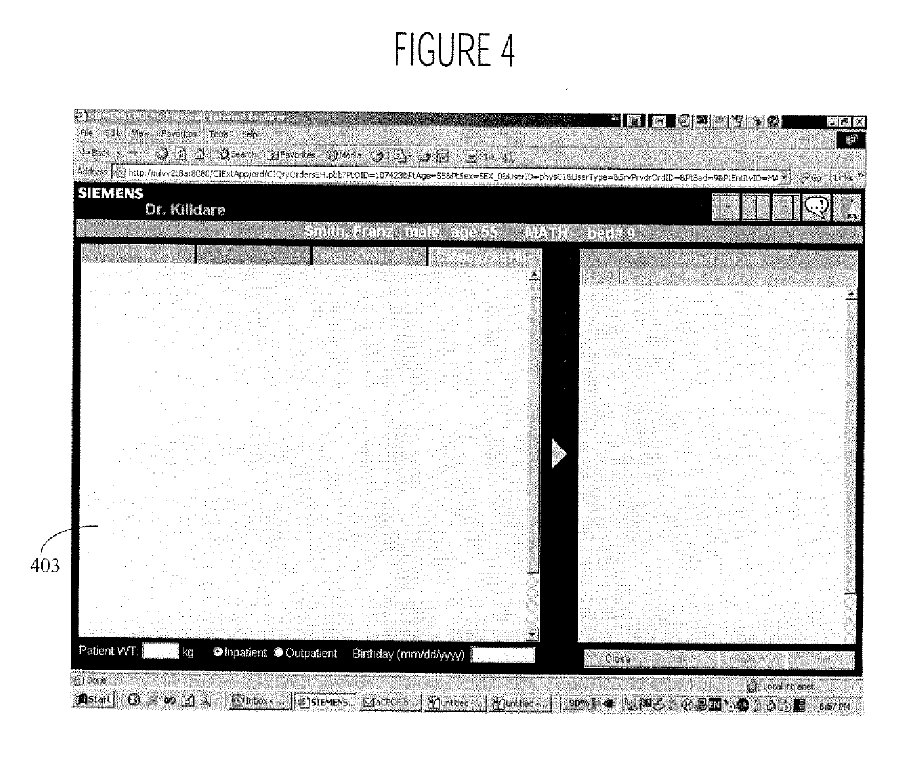

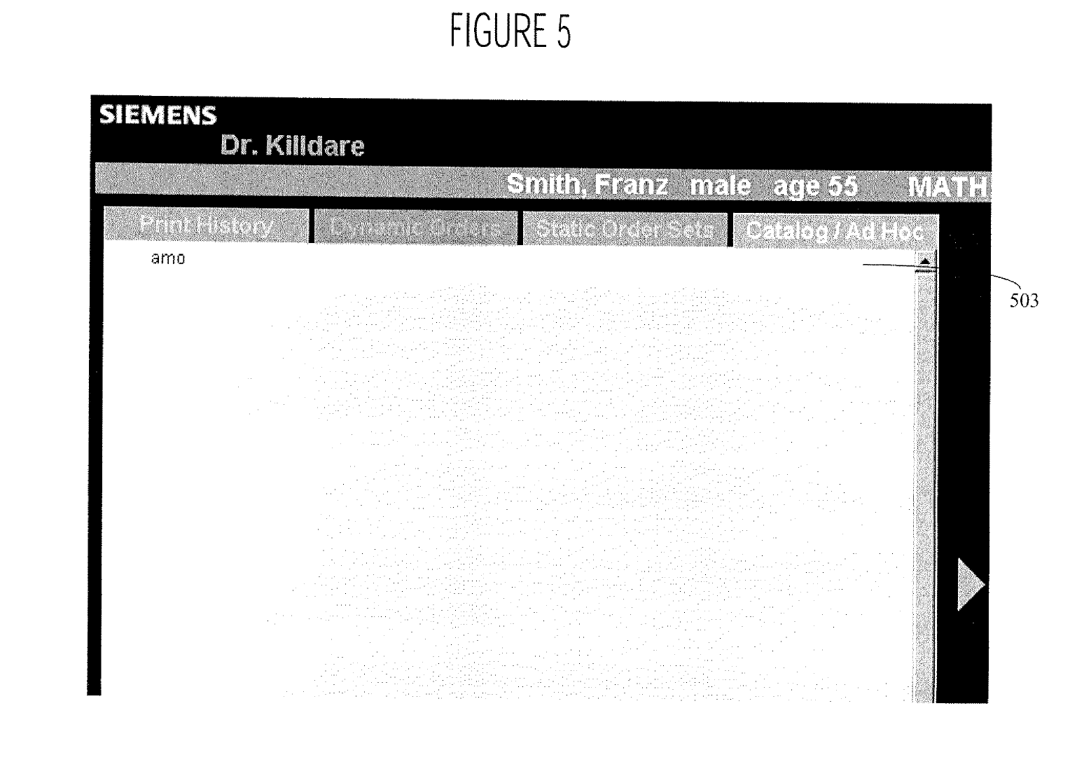

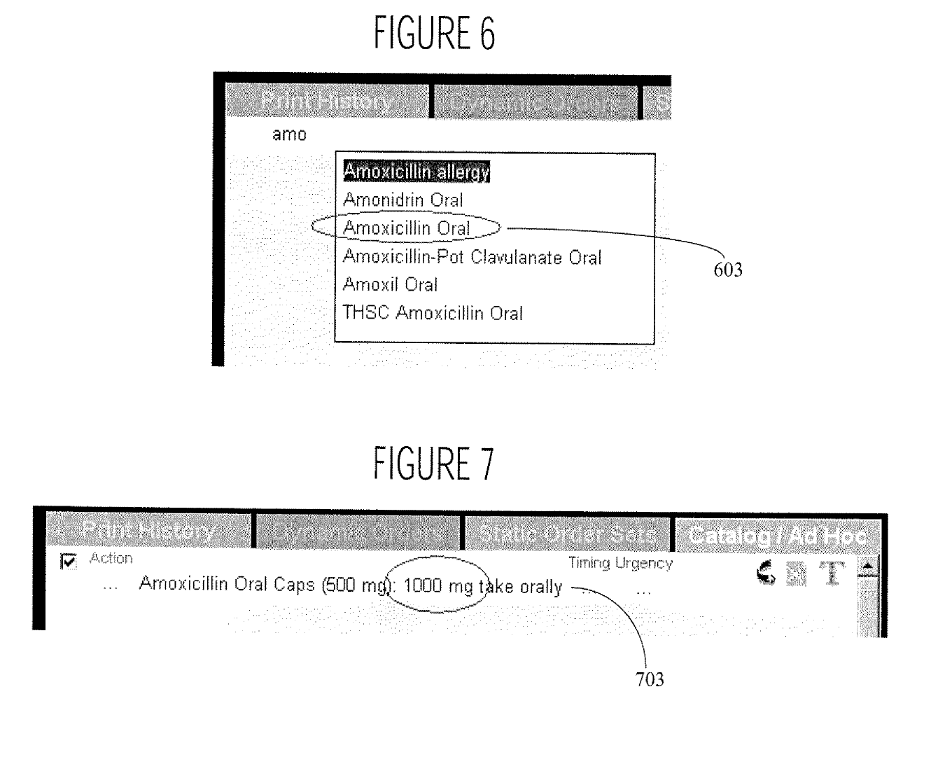

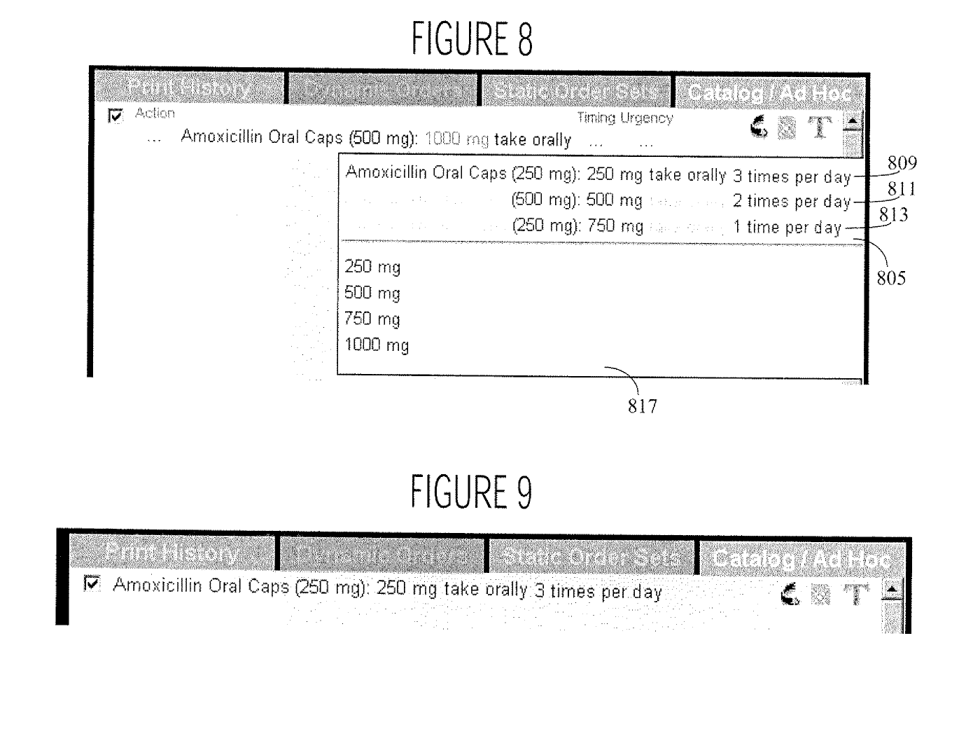

In an example of operation, in response to user command system 1 provides an empty page image menu illustrated in FIG. 4, enabling user selection of patient treatment orders from a Catalog or for entry of adhoc orders in window area 403. A user enters letters “amo” on line 503 as illustrated in FIG. 5. In response system 100 provides, via display images 120, a list of orderable items that have a text string description that contains at least one word that starts with the letters “amo” as illustrated in FIG. 6. In response to user selection of “Amoxicillin Oral” 603, system 100 provides an initial candidate order phrase 703 illustrated in FIG. 7. The user, upon review of order phrase 703, desires to change the 1000 mg value of the drug dosage attribute and clicks on it. In response, system 100 generates up to 5 alternate complete candidate order representative phrases shown in image area 805 of the drop down menu illustrated in FIG. 8. The phrases are complete as far as the user is concerned but from a service provider point of view the order may be incomplete and a clerk or nurse may provide supplementary data such as scheduled start time, etc. The alternate candidate order representative phrases individually contain a value for the attribute selected by the user that is different from and excludes the initial 1000 mg value. (250 mg, 500 mg and 700 mg in items 809, 811 and 813 respectively). System 100 advantageously shows just three complete candidate phrases to keep the reading load for a user reasonably low and to maintain a user friendly feel for the user interface. The list of three items is not exhaustive and many more candidate phrases exist. System 100 generates the three alternate candidate order representative phrases using two heuristics. Specifically, (1) the dosage value should not be 1000 mg (since the user clicked on that value in the initial candidate phrase 703 and it is assumed the user therefore wants a different value) and (2) of the Amoxicillin Oral Caps orders the user has previously placed, the system shows the orders that were placed by the user most often in the past.

An individual candidate order representative phrase may be viewed as a set of attribute values. For example, the attributes may be, drug name (amoxicillin), form (caps), strength (250 mg per capsule), total dose (750 mg), frequency (how often the dose should be taken, e.g., 1 time per day). User selection of a candidate phrase means that the user selects with a single click values in many different attribute value menus. In an alternative embodiment, system 100 selects a different dose (e.g., change 1000 mg to 750 mg) in one menu and select a different frequency (chance “1 time per day” to “2 times per day”) in another menu. System 100 also shows an exhaustive list of values in image window area 817 for an attribute that the user desires to change. If none of the complete candidate phrases in the popup menu in window area 805 is correct, the user can select a new attribute value from the exhaustive list in area 817. In the FIG. 8 example, upon selection of a value in area 817 a user also changes a value of at least one other attribute to obtain the correct target phrase or in another embodiment system 100 does this automatically. FIG. 9 illustrates selected order representative phrase 809 within order placement window 403 of the FIG. 4 ordering menu.

System 100 increases the speed with which a user 107 can specify multiple attributes used by the computer application 128. The heuristics take into account the likelihood that a particular combination of menu items is selected, the reading load that may be experienced by a user 107 when trying to locate the desired menu items, and the time and effort required to scroll large menus, to open multiple menus and to locate and select desired items in those menus. System 100 uses data indicating combination of frequency of selection and recentness of selection.

Returning to FIG. 1, system 100 enables user selection of related parameters identifying an order 132, as shown in FIG. 7, for providing an item 130 (e.g., item 809 FIG. 8). The repository 106 includes information identifying candidate items 130 for order and associated corresponding related order parameters 134. An individual item for order is associated with multiple related order parameters. The user interface processor 122 provides data representing a display image 120 identifying an initial complete candidate order 136 including multiple related parameters, in response to user entry of order-associated data 140. In response to user selection of an individual parameter 144 of the initial complete candidate order 136, the user interface processor 122 provides data representing multiple, different individually user selectable complete candidate orders 148 incorporating corresponding sets of different related order parameters. The complete candidate orders are derived using the repository 106 and based on user ordering history 142. The user interface processor 122 selects one of the multiple, different individually user selectable complete candidate orders 148 in response to a user command 111.

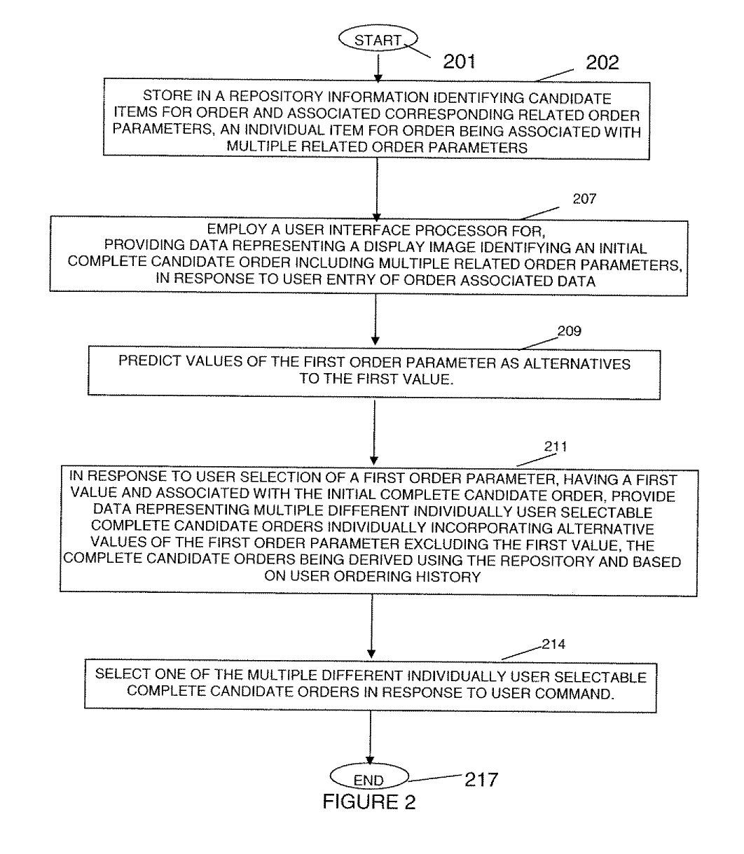

FIG. 2 shows a flowchart of a process performed by system 100 (FIG. 1) supporting ordering of an item. In step 202 following the start at step 201 system 100 stores, in repository 106, information identifying candidate items for order and associated corresponding related order parameters, an individual item for order being associated with multiple related order parameters 138, 140. Processor 104 in step 207 initiates generation of data representing a display image 120 identifying an initial complete candidate order including multiple related order parameters, in response to user entry of order associated data via user interface 102. The initial complete candidate order is a single order derived by identifying an order having the highest probability of being desired by the user based on user prior ordering history and received medical information of the patient concerned, using repository 106. The initial complete candidate order may also be derived based on at least one of, (a) predetermined clinical guidelines, (b) predetermined departmental treatment preferences and (c) treatment resource availability. The orders are orders for providing medical treatment for a patient and the related order parameters identify at least one of, (a) quantity, (b) a route of administration of a medical treatment, (c) a frequency of administering a treatment and (d) a form of medical treatment. The form of medical treatment comprises a package type, a strength of a medical treatment or a concentration of a medical treatment.

In step 209, prediction processor 124 predicts values of the first order parameter as alternatives to the first value. Prediction processor 124 predicts values of the first order parameter related to an individual order based on at least one of, (a) user ordering history, (b) frequency of ordering of an order and (c) clinical logic 150. The clinical logic employs Bayesian logic, Hidden Markov Models, neural networks, first order logic, description logic, fuzzy description logic and/or fuzzy logic. Prediction processor 104 predicts values of the first order parameter related to an individual order based on ordering history of an entity associated with the user and the entity comprises a hospital or a hospital department. The alternative value of the order parameter of the individual order constrains a set of allowable values of the remaining order parameters 152 of the individual order. Prediction processor 124 predicts the initial complete candidate order and the multiple different individually user selectable complete candidate orders 148, based on user entered text partially identifying an order.

In step 211, in response to user selection of a first order parameter, having a first value and associated with the initial complete candidate order processor 104 operating in conjunction with user interface 102, provides data representing multiple different individually user selectable complete candidate orders individually incorporating values of the first order parameter excluding the first value. The complete candidate orders being derived using repository 106 and based on user ordering history 142. A candidate order phrase includes order attribute values in the initial candidate phrase or attribute values explicitly chosen by the user. A user does not need to reevaluate an entire candidate phrase after each attribute value selection. When a user clicks on an attribute value in a current candidate phrase, system 100 displays a menu with a small set of new candidate phrases at the top (or elsewhere in another embodiment). If order attributes are combined in a single menu this way, the resulting menu could contain hundreds or even thousands of candidate phrases. This is undesirable. In contrast, system 100 advantageously limits the menu to a small number of candidate phrases. The multiple different individually user selectable complete candidate orders list is limited and not exhaustive. Processor 104 creates the limited candidate order list without changing attribute values already explicitly chosen by a user when he or she selected an orderable services from the search result list (the list that appears after the user enters a few letters like ‘amo’) and/or in previous interactions with the candidate phrase. In addition, candidate phrases of the limited list individually contain different values for an attribute clicked on (and selected) by a user in an initial candidate order since system 100 assumes a user clicked on the original attribute value in order to chance it. In response to the previous two restrictions, system 100 shows the top n (e.g. top 5) order phrases most often used by either a current user or an organization. Further, because system 100 presents the limited candidate order list in a menu, a user already has an expectation that each candidate phrase is to be evaluated in its entirety, in contrast to candidate phrases displayed when no menu is visible.

User interface processor 122 provides data representing a display image 146 identifying, the initial complete candidate order 136 (e.g. as illustrated in window area 805 of FIG. 8) and the multiple different individually user selectable complete candidate orders 148 individually incorporating alternative values of the first order parameter excluding the first value together with the alternative values (e.g. as illustrated in window area 817 of FIG. 8). The alternative values in window area 817 are a substantially exhaustive list of available alternative values for the candidate order attribute the user clicked on in an initial candidate order phrase (e.g. as illustrated in window area 703 of FIG. 7). The relative position of the list of available alternative values is arbitrary and it may be above or below the candidate orders, for example and may be represented in a different manner such as a gauge, slider, interactive graph, calendar, clock, table or any other representation enabling user selection of a desired attribute value. For some order parameters the system allows the user to enter his own value or values for that parameter. In step 214, processor 104 with user interface 102, selects one of the multiple different individually user selectable complete candidate orders in response to user command. The process of FIG. 2 terminates at step 217.

System 100 reduces the average total time to create an order set or to complete a document by reducing the number of user interactions and by reducing the need for a user to read and evaluate intermediate results generated by the system. An alternative embodiment advantageously creates a similar result by presenting candidate sets of related attribute values in one or more rows or one or more columns in a grid, table or spreadsheet. A user can make a single selection of the row/rows or column/columns to achieve the desired set of attribute values. The system is usable in user interface option selection and specifically, in Clinical Order Entry and clinical documentation by clinicians such as physicians, nurses, therapists, for example.

The system, processes and menus presented in FIGS. 1-9 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. A system according to invention principles is applicable to order management in healthcare and other fields. Further, any of the functions provided in the systems of FIGS. 1-2 may be implemented in hardware, software or a combination of both and may reside on one or more processing devices located at any location of a network linking the FIG. 1 elements or another linked network including another intra-net or the Internet.

What is claimed is:

1. A user interface system enabling user selection of related order parameters identifying an order for providing an item or service, comprising
a repository including information identifying candidate items for an order and associated corresponding related order parameters, an individual item for an order being associated with a plurality of related order parameters; and
a user interface processor for providing data representing a display image identifying an initial complete candidate order including a plurality of related order parameters, in response to user entry of order associated data, in response to user selection of a first order parameter, having a first value and associated with said initial complete candidate order, providing data representing a plurality of different individually user selectable complete candidate orders individually incorporating values of said first order parameter excluding said first value, said complete candidate orders being derived using said repository and based on user ordering history and
selecting one of said plurality of different individually user selectable complete candidate orders in response to user command.

2. A system according to claim 1, wherein
said initial complete candidate order is a single order and said orders are orders for providing services directly or indirectly associated with the medical treatment of a patient.

3. A system according to claim 2, wherein
said initial complete candidate order is a single order and
said orders are orders for providing medical treatment for a patient and said related order parameters identify at least one of, (a) quantity, (b) a route of administration of a medical treatment, (c) a frequency of administering a treatment and (d) a form of medical treatment.

4. A system according to claim 3, wherein
said form of medical treatment comprises at least one of, (a) a package type, (b) a strength of a medical treatment and (c) a concentration of a medical treatment.

5. A system according to claim 1, including
a prediction processor for predicting values of said first order parameter as alternatives to said first value.

6. A system according to claim 5, wherein
said prediction processor predicts values of said first order parameter related to an individual order based on at least one of, (a) user ordering history, (b) frequency of ordering of an order and (c) clinical logic.

7. A system according to claim 6, wherein
said clinical logic employs at least one of (a) Bayesian logic, (b) Hidden Markov Models, (c) neural networks (d) fuzzy logic, (e) first order logic, (f) description logic, and (g) fuzzy description logic

8. A system according to claim 5, wherein
said prediction processor predicts values of said first order parameter related to an individual order based on ordering history of an entity associated with said user and
said entity comprises at least one of, (a) a hospital and (b) a hospital department.

9. A system according to claim 5, wherein
said user interface processor provides data representing a display image identifying,
said initial complete candidate order,
said plurality of different individually user selectable complete candidate orders individually incorporating alternative values of said first order parameter excluding said first value and
a plurality of said alternative values.

10. A system according to claim 9, wherein
said plurality of said alternative values is a substantially exhaustive list of available alternative values.

11. A system according to claim 5, wherein
said prediction processor predicts alternative values of said first order parameter and
said alternative value of said order parameter of said individual order constrains a set of allowable values of said remaining order parameters of said individual order.

12. A system according to claim 1, wherein
said initial complete candidate order is derived by identifying an order having the highest probability of being desired by the user based on user prior ordering history or ordering history of user peers and received medical information of the patient concerned, using said repository.

13. A system according to claim 1, wherein
said initial complete candidate order is derived based on at least one of, (a) predetermined clinical guidelines, (b) predetermined departmental treatment preferences and (c) service provider resource availability.

14. A system according to claim 1, including
a prediction processor for predicting at least one of, (a) said initial complete candidate order and (b) said plurality of different individually user selectable complete candidate orders, based on user entered text partially identifying an order.

15. A user interface system enabling user selection of related order parameters identifying an order for providing an item, comprising:
a repository including information identifying candidate items for order and associated corresponding related order parameters, an individual item for order being associated with a plurality of related order parameters; and
a user interface processor for,
providing data representing a display image identifying an initial complete candidate order including a plurality of related order parameters, in response to user entry of order associated data,
in response to user selection of a first order parameter, having a first value and associated with said initial complete candidate order, providing data representing a plurality of different individually user selectable complete candidate orders individually incorporating alternative values of said first order parameter excluding said first value, said complete candidate orders being derived using said repository and based on user ordering history and
selecting one of said plurality of different individually user selectable complete candidate orders in response to user command.

16. A method enabling user selection of related order parameters identifying an order for providing an item, comprising the activities of:
storing information identifying candidate items for order and associated corresponding related order parameters, an individual item for order being associated with a plurality of related order parameters;
providing data representing a display image identifying an initial complete candidate order including a plurality of related order parameters, in response to user entry of order associated data;
in response to user selection of a first order parameter, having a first value and associated with said initial complete candidate order, providing data representing a plurality of different individually user selectable complete candidate orders individually incorporating values of said first order parameter excluding said first value, said complete candidate orders being derived using said repository and based on user ordering history; and
selecting one of said plurality of different individually user selectable complete candidate orders in response to user command.
